(19) United States
(12) Patent Application Publication
(10) Pub. No.: US 2026/0355545 A1
(43) Pub. Date: Nov. 12, 2026

(54) System and Method for Ultrasound Tomography Guided Localized Mild Hyperthermia (MHTh) Using a Common Closed Geometry Transducer

(71) Applicant: University of Rochester, Rochester, NY (US)

(72) Inventors: Mohammad Mehrmohammadi, Rochester, NY (US); Nebojsa Duric, Rochester, NY (US); Peter John Littrup, Bloomfield Hills, MI (US)

(73) Assignee: University of Rochester, Rochester, NY (US)

(21) Appl. No.: 19/157,185

(22) PCT Filed: Feb. 16, 2024
(86) PCT No.: PCT/US2024/016142

§ 371 Date: Aug. 15, 2025

Related U.S. Application Data
(60) Provisional application No. 63/446,616, filed on Feb. 17, 2023.

(51) Int. Cl. A61N 7/02; A61N 7/00

(52) U.S. Cl. CPC A61N 7/02; A61N 2007/0052; A61N 2007/0078

(57) ABSTRACT

A system and method for ultrasound tomography guided localized mild hyperthermia using a common closed geometry transducer is disclosed. The system has an imaging modality, a thermometry modality and a therapeutic modality which all use a closed geometry transducer in common. Methods of the present invention include imaging with ultrasound tomography, therapeutically increasing temperature of target tissue using ultrasound heating, monitoring the increase in temperature using ultrasound based thermometry, and removing the therapeutic ultrasound signals once a specified temperature is achieved, where the method is performed with a common platform and closed geometry transducer.

### CROSS REFERENCE TO RELATED PATENT APPLICATIONS

This application is a 371 entry of PCT/US2024/016142 filed on Feb. 16, 2024 entitled “System and Method for Ultrasound Tomography Guided Localized Mild Hyperthermia (MHTh) Using a Common Closed Geometry Transducer” by Mehrmohammadi et al., and claims priority to U.S. Patent Application Ser. No. 63/446,616 filed on Feb. 17, 2023 entitled “System and Method for Ultrasound Tomography Guided Localized Mild Hyperthermia Using a Common Closed Geometry Transducer” by Mehrmohammadi et al., the entire disclosures of which are incorporated herein by reference.

### STATEMENT REGARDING FEDERALLY SPONSORED RESEARCH OR DEVELOPMENT

None

### TECHNICAL FIELD

The present invention relates generally to ultrasound tomography, and more particularly to a system and method for ultrasound tomography guided localized mild hyperthermia using a common closed geometry transducer.

### BACKGROUND ART

Breast cancer (BrCa) is the most common cancer among U.S. women and the second leading cause of cancer death in this population. It is estimated that more than 287,850 new cases of invasive BrCa have been diagnosed in 2022, leading to the death of more than 43,000 American women. Treatment choices for BrCa patients include surgery, hormonal therapy, molecular targeted therapies, chemotherapy (ChT), radiation therapy (RT), and immunotherapy (ImT). They are often used in combination to address the local, regional and systemic extent of cancer, while also attempting to minimize their overall side effect profiles. For most localized tumors, surgical procedures are the standard of care, but minimally invasive treatments are being sought for all stages of cancer. ChT is often used to shrink large tumors before surgery to reduce complications from surgery and to minimize disfigurement from BrCa surgery. However, limitations of chemotherapy include damage to proliferating healthy cells due to its systemic delivery and the development of innate and/or acquired drug resistance. RT for early-stage breast cancer decreases recurrence rates and improves breast cancer-specific survival for most patients. However, long-term follow-up studies have reported treatment toxicities after RT, such as an increased risk of heart disease, which has been attributed to incidental irradiation outside the target volumes. ImT is typically recommended for patients in advanced stages of BrCa to control the disease by boosting the patients' immune system to better recognize and destroy cancer cells. While ImT shows promising benefits and increases the survival in BrCa patients, primary and acquired resistance to the treatment limits its efficacy in some patients. Enhancing the efficacy of these BrCa treatment methods can impact the quality of patient care by improving the treatment outcomes and reducing the discomfort of side effects. The National Cancer Institute (NCI) recognizes such adverse treatment effects as a vital survivorship issue needing further research.

Mild Hyperthermia (MHTh), a method by which a tumor is selectively heated to increase its tissue temperature (~42 °C, or ΔT≤6 °C) has shown promises in enhancing ChT, RT, and ImT. MHTh has been shown to provide a platform for more cancer treatment by: (a) increasing a drug uptake and thus reducing the dosage requirements; (b) induction of heat shock proteins in tumor cells leading to an anticancer immune response and increased cytotoxicity of drugs; (c) increasing the tumor oxygenation, leading to enhanced radiation response; (d) inhibiting and delaying the repair of RT-induced DNA damage, thus increasing the efficacy of RT; (e) promoting both active and passive release of tumor antigens; (f) enhancing immune surveillance by T-cells; and several other pathways that are widely studied in the literature. MHTh can also be utilized for targeted cancer treatment by providing an opportunity for controlled release of chemotherapeutic (and immunogenic) agents through utilizing thermosensitive liposomes such as FDA-approved Thermodox®. MHTh has also been studied as a stand-alone treatment of cancer, although it is mostly known as a booster to ChT, RT, and ImT.

MHTh can be induced locally, regionally, and throughout the entire body. Local MHTh is highly desirable because it does not cause damage to the healthy tissue, cause bleeding or blood clots, or cause other side effects such as diarrhea and nausea. MHTh generation systems include both invasive and non-invasive methods. Focused Ultrasound (FUS), radiofrequency (RF) applicators, and microwave (MW) tools are often used to induce MHTh. During the MHTh procedure, accurate adjustment and monitoring of tissue temperature is critical to maintain tissue temperatures within the desired narrow range (typically 41-43 °C). Therefore, real-time temperature monitoring (or thermometry) is an essential need to ensure the success of MHTh-assisted treatments. The inability to precisely heat a deep-seated tumor, and monitor changes in temperature within the tumor, within the optimal range of maximal benefit has thwarted the translation of hyperthermia as an effective clinical treatment modality. Noninvasive thermometry has been favored over invasive methods using externally inserted temperature sensors, which involves magnetic resonance imaging (MRI) and Ultrasound (US). MRI thermometry is capable of measuring both absolute temperature and temperature changes. However, MRI thermometry suffers from low temporal resolution, and choosing MRI as a temperature sensing and guidance method adds to the complexity and cost of the hyperthermia delivery system as well as reducing accessibility. US thermometry, often relying on the changes of sound speed (SS) as a result of temperature variation, has demonstrated great potential for tissue temperature measurement and monitoring. However, US thermometry using traditional linear phased array transducers does not measure SS directly and therefore performs poorly in highly heterogenous tissues such as the breast. Furthermore, the absence of direct SS measurements negatively impacts on precise focusing of acoustic energy onto the tumor through time-reversal (TR) techniques.

Recently, the FDA approved Ultrasound tomography (UST) for screening women with dense breast tissue. The system was developed as an operator-independent, volumetric imaging modality for breast cancer imaging, using a ring-shaped US transducer. UST not only uses information from reflected signals, but also transmission signals by incorporating piezoelectric elements on opposing sides of the tissue of interest. Consequently, it can measure other diagnostically relevant parameters such as the sound speed (SS) and acoustic attenuation (AA). SS and AA information can be used to characterize tissue composition as well as biomarkers such as the stiffness (similar to elasticity) of the breast tissue. SS images are created by extracting the transmitted signals between emitter-receiver pairs and using waveform inversion methods to reconstruct images. These SS images can then be converted to temperature maps given the relation between SS and temperature rate of change of ~2 m/s/°C in the range of 25-42 °C, depending upon tissue type. The ability to measure SS directly, at sub-mm resolution, allows for accurate focusing of energy and accurate temperature monitoring, thereby enabling, for the first time, the ability to interleave imaging and treatment using only the single transducer.

While MHTh has been studied extensively and is shown to significantly enhance cancer treatment, it is not yet widely used in clinical practice. Major technical obstacles and difficulties including lack of optimum and satisfactory localized heating profiles; complexity (and cost) of existing systems; lack, inaccuracy, or technical difficulties to develop temperature monitoring, has limited its clinical utility. According to NCI, one major drawback of MHTh is the need for special equipment and expertise, which is not widely available. This had prevented further studies and translation of MHTh into patient care systems. Therefore, there is an unmet clinical need for a non-invasive, safe, cost effective and easy to implement system that can provide fully controlled localized heating profiles in targeted lesions (i.e., tumors).

What is therefore needed is a system and method for mild hyperthermia treatment that contains an imaging modality, a thermometry modality, and a therapeutic modality using a single platform and transducer configuration.

### DISCLOSURE OF THE INVENTION

In accordance with the present invention, there is provided a system for ultrasound-guided localized mild hyperthermia comprising a closed geometry transducer; an imaging modality, a thermometry modality, and a therapeutic modality; wherein the imaging modality, the thermometry modality and the therapeutic modality use the closed geometry transducer in common. Methods for ultrasound-guided localized mild hyperthermia using a common closed geometry transducer are also provided.

The foregoing has been provided by way of introduction, and is not intended to limit the scope of the invention as described by this specification and the attached drawings.

### BRIEF DESCRIPTION OF THE DRAWINGS

The invention will be described by reference to the following drawings, in which like numerals refer to like elements, and in which:

FIG. 1a depicts a UST scanner;

FIG. 1b depicts coronal UST images;

FIG. 1c depicts a system of the present invention;

FIG. 2a depicts a UST thermometry system;

FIG. 2b shows a UST thermometry system in use;

FIG. 2c shows SS images at various temperatures;

FIG. 2d is a graph of average SS and standard deviations at inclusions vs. temperature;

FIG. 3a depicts time reversal with aberration correction;

FIG. 3b depicts focal pressure profiles;

FIG. 3c is a qualitative comparison of pressure localization;

FIG. 4a depicts TR focusing with a uniform sound speed;

FIG. 4b depicts UST assisted TR focusing;

FIG. 4c illustrates focusing with no compensation;

FIG. 4d illustrates focusing with SOS compensation;

FIG. 5a is a map of maximum absolute pressure over a range of positions r;

FIG. 5b is a temperature map using the measured absolute pressure with a tuning duty cycle and bioheat simulation;

FIG. 6a depicts a hydrophone measured pressure field generated by a phased array transducer;

FIG. 6b depicts a simulated pressure field generated by a phased array transducer;

FIG. 6c shows an experimental setup to measure temperature rise in bovine muscle;

FIG. 6d is a graph showing the experimentally measured temperature rise from the experimental setup in FIG. 6C;

FIG. 7a depicts in-silico MHTh generation results;

FIG. 7b depicts further in-silico MHTh generation results;

FIG. 7c depicts further in-silico MHTh generation results;

FIG. 7d depicts further in-silico MHTh generation results;

FIG. 7e depicts further in-silico MHTh generation results;

FIG. 7f depicts further in-silico MHTh generation results;

FIG. 8 depicts UST images with varying frequencies and element count;

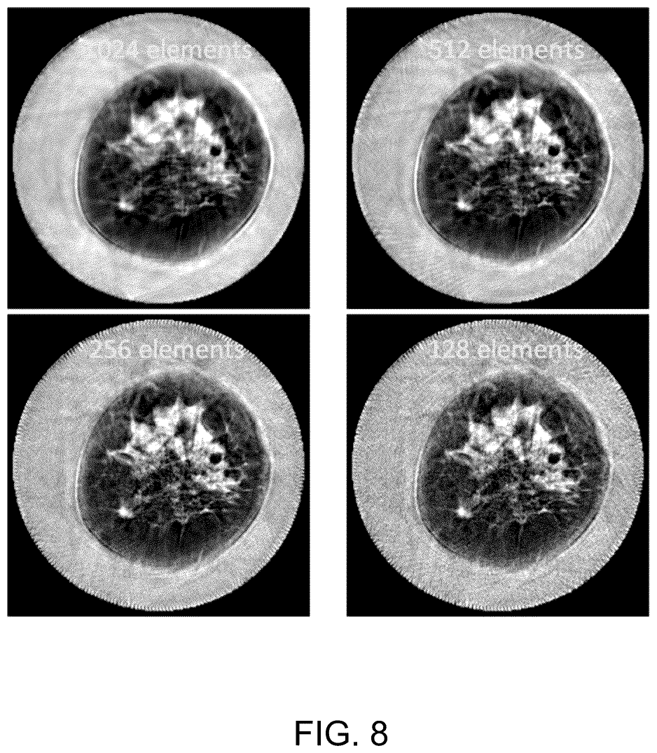

FIG. 9 is a schematic diagram of UST-guided MHTh treatment;

FIG. 10a depicts a 3D vascular map with raw blood flow signal intensity;

FIG. 10b is a graph of blood SO₂ measure by sPA vs. blood gas analysis;

FIG. 11a is a graph of Doxil uptake in MHTh treated vs. untreated tumors;

FIG. 11b shows the statistical difference in uptake between treated and control 4T1 tumors; and

FIG. 11c illustrates tumor volume as a function of time for 4T1 tumors treated with 8Gyx3 at varying doses of immunotherapy.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification and drawings attached hereto.

### BEST MODE FOR CARRYING OUT THE INVENTION

To address the unmet clinical need previously described, the following discloses an all-acoustic image-guided MHTh theranostic system. The disclosed system has several innovative features that differentiate it from systems using conventional Ultrasound (US). These innovative features are summarized in Table 1.

TABLE 1
Innovative aspects of UST-guided MHTh system with ring-array transducer

| Innovative Feature | Technical advantage | Clinical implications |
|---|---|---|
| Ring array transducer | Isotropic tissue coverage for imaging and | Avoids non-uniform tissue coverage inherent |
| | heating, synergistic combination of UST imaging | to conventional transducers (sidelobes, etc). |
| | and MHTh. | |
| Accurate acoustic focusing | Fast and accurate beamforming for imaging and | High contrast imaging and temperature |
| for MHTh generation | heating, possibility of using UST information for | modulation of tissues that satisfies clinical |
| | aberration-free acoustic focusing. | criteria such as treatment time. |
| Transmission US capability | Direct SS maps for accurate aberration | Improved heating profile to avoid damage to |
| | corrections and better focusing. Better tracking of | healthy tissues. |
| | SS changes for non-invasive temperature | |
| | monitoring and control. | |
| Integration of UST and | Imaging and heating performed by same | No need for ancillary equipment and complex |
| MHTh | transducer using interleaved approach. | patient procedures in specialized suites, easy |
| | Compactness and portability, and availability for | to adopt by physicians and technicians |
| | the patients | |
| Low cost and practical | Inexpensive US components with no need for | Increase availability for all clinics. |
| solution | complex US transceiver circuit | |
| FDA approved platform | Building around (as an adjunct to) a commercially | Increase access and insurance coverage. |
| | available device facilitates future approvals. | |

A ring US transducer or other closed geometry transducer provides an ideal platform to induce localized heating by applying TR principles and using multiple low (as compared to HIFU) energy acoustic transmission to generate heat in targeted lesions of different depths. The synergistic combination of UST imaging and MHTh induction provides a unique opportunity to use UST-driven a priori information of tissue composition for accurate TR focusing and heat induction. TR focusing uses multiple lower-energy acoustic transmissions on a ring geometry to converge upon the target allowing for reduction of unnecessary heat in surrounding normal tissues (and especially on tissue surfaces) while also allowing for faster deposition of necessary thermal doses. In addition, the combination of tissue composition and acquired SS maps, driven by UST, provides a platform for real-time thermometry. In summary, enabling a closed geometry system such as a ring-array US system to perform both MHTh generation and tomographic temperature monitoring provides a straightforward pathway for enhancing BrCa treatment outcomes. The disclosed system is non-invasive and non-ionizing, which eliminates the ionizing radiation concern, and will be widely available to BrCa patients and at a low cost compared to existing competing technologies.

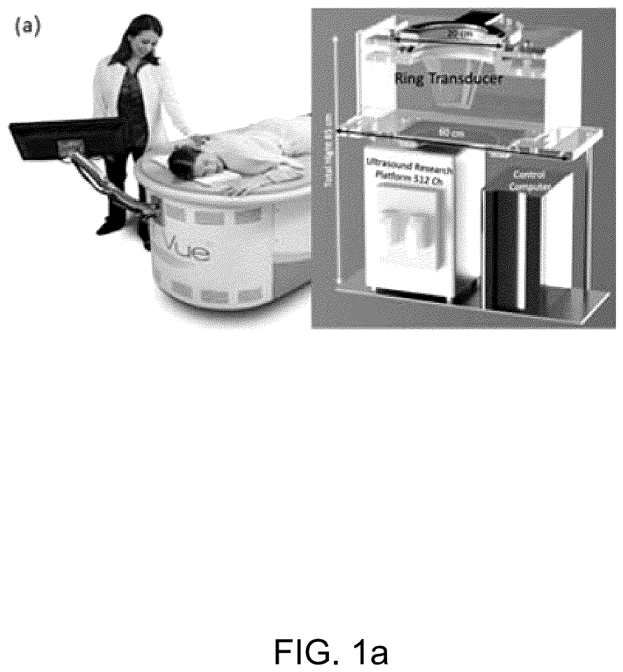

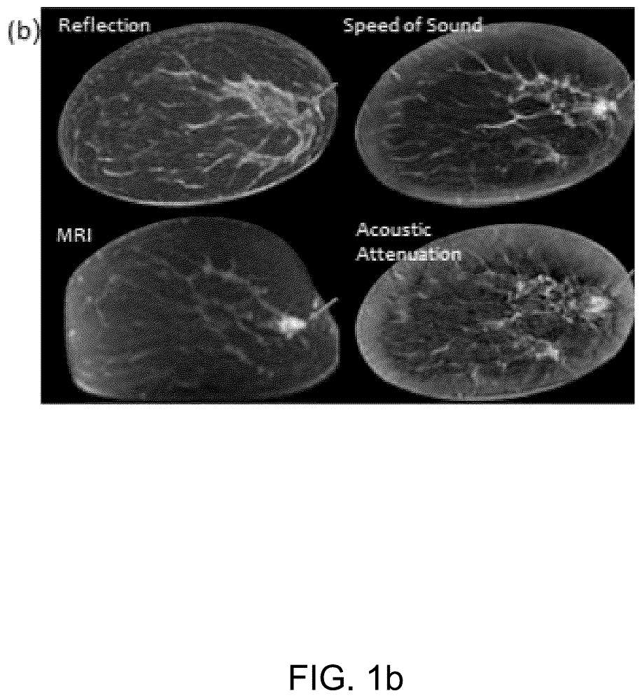

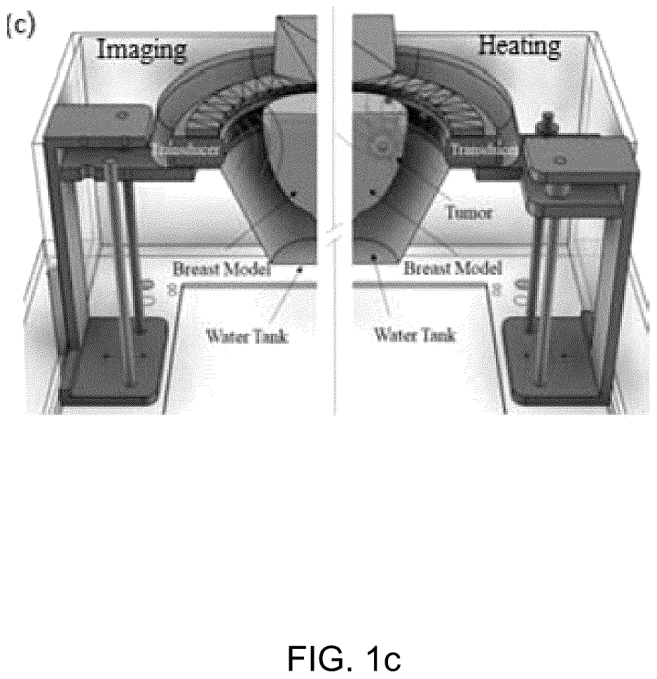

Turning to FIG. 1, (a) FDA-approved clinical UST scanner (SoftVue®, Delphinus Medical Technologies) which uses a ring-shaped US transducer to acquire volumetric reflection and transmission derived acoustic maps of tissue. (b) coronal UST images of a patient with a 9 mm invasive ductal carcinoma compared with corresponding contrast enhanced MRI (bottom row). (d) Schematic of the ring-based UST-guided MHTh theranostic system of the present invention. For the sake of better visualization, on the left UST SS imaging through sequential element transmission and reception is depicted, and the right half shows the disclosed targeted heating using modified acoustic emission (transmit waveform) from the full ring. The system operates in time sequenced hyperthermia/imaging regimen.

A system for ultrasound-guided localized mild hyperthermia is disclosed that comprises a closed geometry transducer; an imaging modality, a thermometry modality, and a therapeutic modality; wherein the imaging modality, the thermometry modality and the therapeutic modality use the closed geometry transducer in common. In some embodiments of the present invention, the closed geometry transducer is a ring transducer. The therapeutic modality comprises the application of therapeutic acoustic signals to target tissue of a patient wherein the therapeutic acoustic signals are configured to cause heating of a specified portion of the target tissue of the patient.

Each of the disclosed modalities (imaging, thermometry and therapeutic) are performed by the components that are further described and depicted herein. Thus, the system for ultrasound-guided localized mild hyperthermia comprises a closed geometry transducer, an imaging component, a thermometry component, and a therapeutic component; wherein the imaging component, the thermometry component and the therapeutic component use the closed geometry transducer in common.

A method for ultrasound-guided localized mild hyperthermia using a closed geometry transducer is further disclosed where the method comprises the steps of propogating diagnostic acoustic signals through target tissue of a patient with a closed geometry transducer; receiving from the closed geometry ultrasound transducer returned acoustic signals from the target tissue of the patient; creating with a processor images of the target tissue from the returned acoustic signals; applying therapeutic acoustic signals to a specified portion of the target tissue with the closed geometry transducer; increasing the temperature of the target tissue with the applied therapeutic acoustic signals; monitoring the increase in temperature of the target tissue with ultrasound thermometry using the closed geometry ultrasound transducer; continuing to increase the temperature of the target tissue and monitor the increase in temperature of the target tissue until a specified temperature is achieved; and removing the therapeutic acoustic signals from the target tissue once the specified temperature is achieved. In some embodiments of the present invention, the method steps are embodied in computer software that operates on computer having a processor, memory, and access to computer readable media.

All experimental studies presented here were performed using a 256-element ring, 200 mm diameter US transducer (Sound Technology Inc., State College, PA) where the center frequency is 1.5 MHz and the bandwidth is 60%. The ring array has a 2.45 mm element pitch and a 9 mm element height. For in-silico studies, k-wave toolbox (MATLAB®, Mathworks, Natick, Massachusetts, USA) were used for both thermal and acoustic forward modeling.

Principles of fast UST thermometry: Temperature dependence of sound speed (SS) in soft tissue has been studied for decades. Significant changes in sound speed, as temperatures increase, have been well documented, including a unique differential heating response from fat. Breast fat also has significantly different sound speed from all other breast tissues, suggesting the eminent feasibility of fat-subtraction imaging utilized with MRI. The imaging and temperature monitoring technology of the present invention employs the newly FDA approved modality of UST (FIG. 1a). UST introduces new image acquisition sequences that allow volumetric assessments of tissue composition (FIG. 1b), including SS (FIG. 1c), and thus tissue temperature, while leveraging its low cost and patient comfort. UST addresses the clinical need for a system that combines the fast imaging, acceptable spatial resolution, and cost-effectiveness of US with the operator independence and physiological volumetric imaging of MRI. Waveform inversion in UST provides high-resolution diagnostic-quality images of SS in breast tissue with high quality structural images that render the parenchymal patter of the breast similarly to contrast MRI (FIG. 1c). An experimental system that embodies these principles is shown in FIG. 1d.

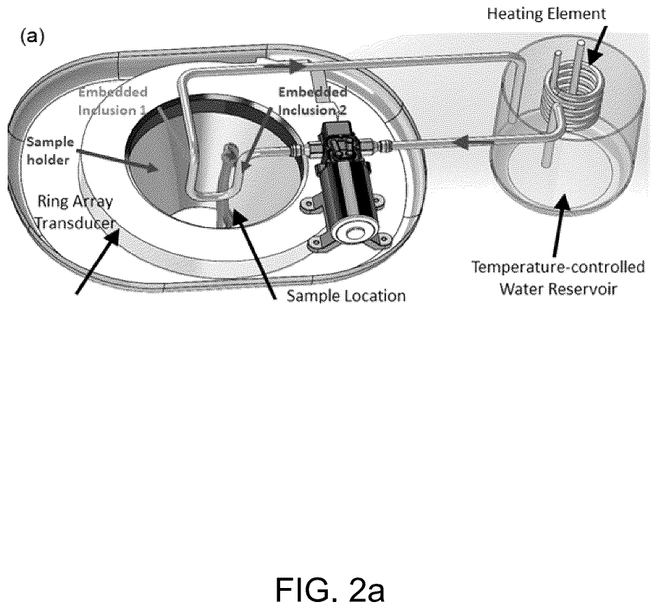

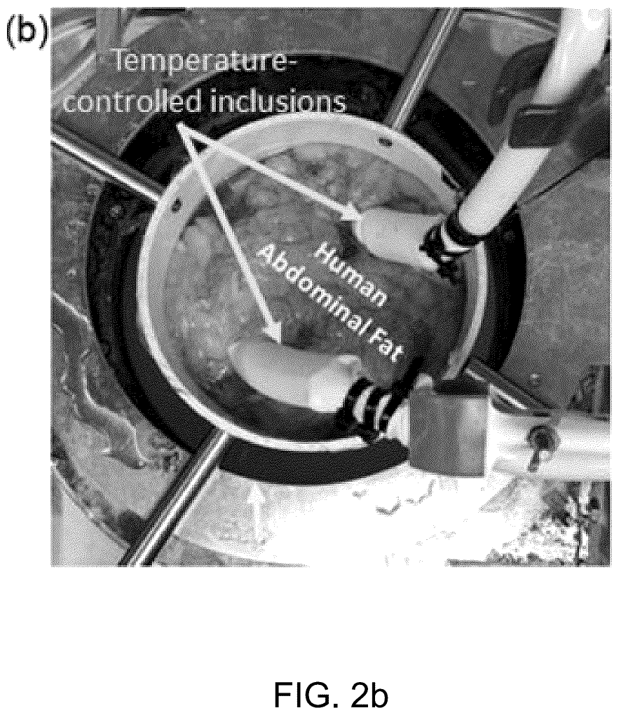

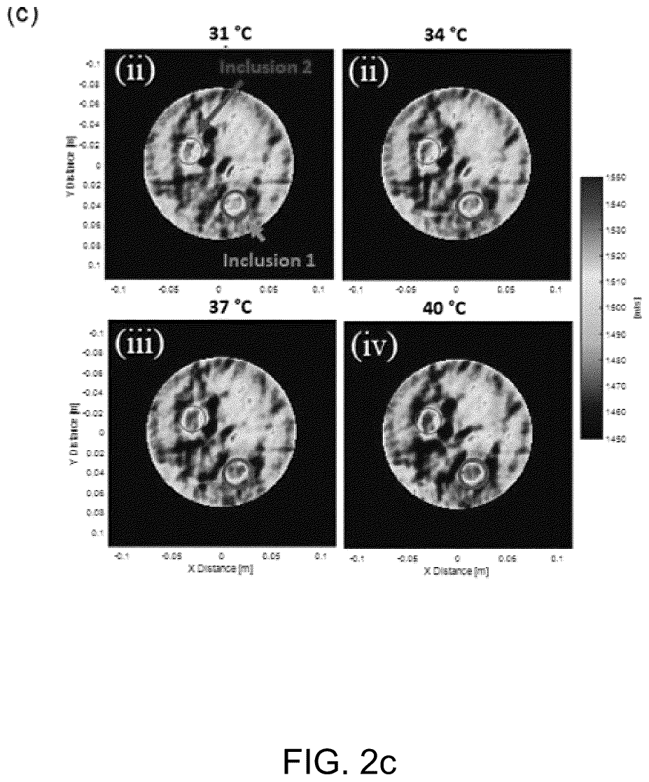

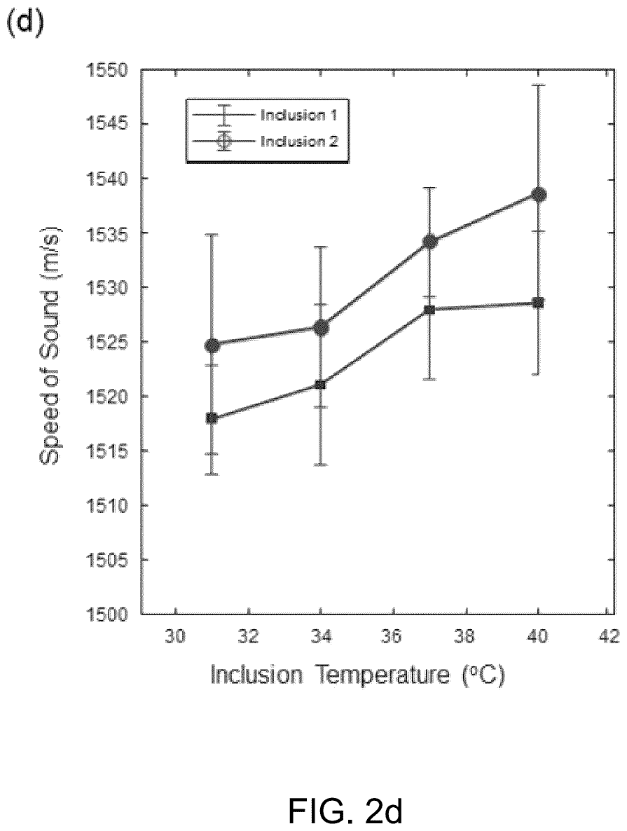

FIG. 2 depicts UST thermometry. (a) Schematic of system showing location of the transducer, phantom, and method for heated water inclusions. (b) Photograph of breast fat mimic phantom made from human fat with embedded tubes containing flowing temperature-controlled water. (c) SS images at all temperatures (i) 31 °C, (ii) 34 °C, (iii) 37 °C, and (iv) 40°; where inclusion 1 is purple and inclusion 2 is red. (d) Average SS and standard deviations at the inclusions vs. temperature.

UST thermometry-Preliminary data: Breast mimicking phantoms were imaged, both of which contained two latex tubes (Medline DYND50423, USA) of 15 mm diameter carrying temperature-controlled water and used a coupling medium of water. The phantoms were made out of human abdominal fat to mimic a scenario of fatty breast tissue. The temperature of the flowing water was controlled using a pump, a temperature controller, and a metal heating element. A schematic of the UST experimental setup and the phantom can be seen in FIGS. 2a&b. The phantom was imaged with the flowing water being maintained between the ranges of 31 °C (room temperature) to 40 °C (close to upper limit for MHTh). FIG. 2 shows the reconstructed SS images at different water inclusion temperatures, in which there is a visually evident increase in the SS at the tube locations compared to the background. To further analyze the SS measurements, mean values along with the SS standard deviations of SS maps, averaged over the imaged volume within both tube locations at all temperature values is shown in FIG. 2d. The SS value increments are in agreement with reported literature. The large standard deviations are due to averaging over the whole tube area as well as repeating multiple measurements (x3), which can cause variations in water temperature flowing inside the phantom. SS measurements in second inclusion is relatively lower than the first inclusion. This was due to the fact that the water flow was from the heating reservoir to inclusion 1 (FIG. 2a), where it then flows through a pipe into the second inclusion and then returns back to the temperature controlled reservoir. We anticipate water cool down during the circulation; i.e. the water flow in inclusion 2 has a lower temperature. Spatial averaging (due to a high contrast SS in inclusions and background may have also affected these results. Knowing the spatial resolution of UST SS measurement, we will further study (Aim 1) the thermometry limitations imposed by UST spatial resolution and we will seek for advanced compensation method by using the point-spread-function analysis of UST SS measurement.

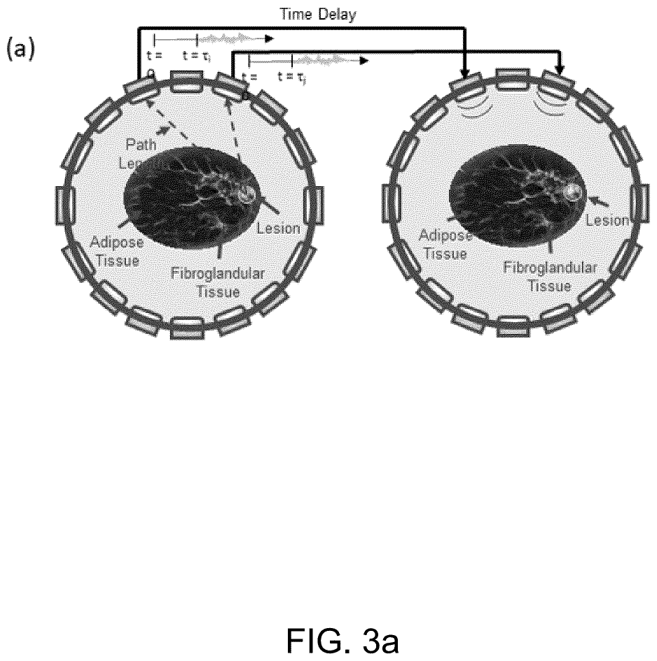

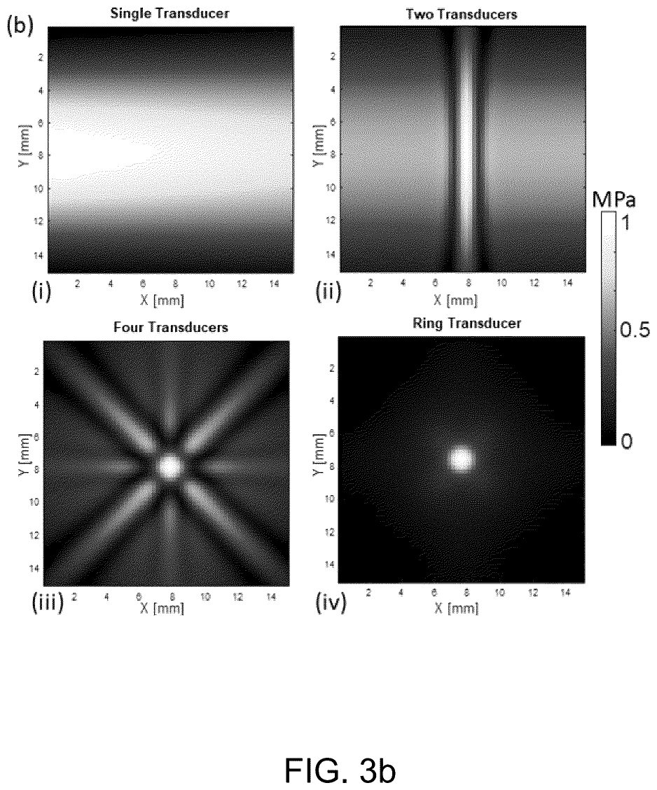

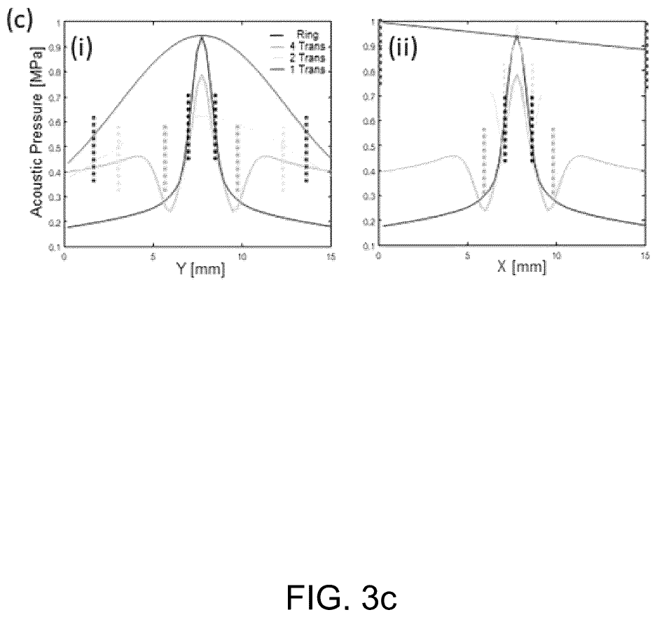

FIG. 3 depicts (a) Time-reversal with aberration compensation; (b) focal pressure profiles using a single, double and four linear transducers (i-iii) and a full-ring array transducer (iv). (c) qualitative comparison of pressure localization through horizontal and vertical in plane focal size analysis.

Feasibility of MHTh generation using a ring-array transducer-In-silico studies: Focusing acoustic energy to generate heat in tissue and by using time-reversal (TR) techniques (FIG. 3a) has been widely studied for different array transducers. TR focusing uses multiple lower-energy transducer beams to converge upon the target, deep inside the tissue. In this manner, risks to intervening tissue are markedly reduced by the distribution of multiple lower power sources using selected paths, allowing more rapid pulse delivery and shorter total treatment times. Heat generation using US is due to conversion of mechanical energy to heat and can be calculated by knowing the distribution of field intensity and the frequency-dependent absorption coefficient of tissue. Using a full-ring array US transducer, allows for a significantly better acoustic focusing (localization, less lobes, and uniformity) and thus more targeted and controllable heating. FIG. 3b, compares the normalized acoustic pressure generated by a one, two and four linear array transducers (3.b i-iii), compared to a full-ring array transducer (3.b iv) have the same number of elements as in a 4 linear transducer arrangement (512 elements). In the case of two and four transducers, they were placed 20 cm (same as the ring diameter) apart from each other and transducers were placed facing each other (one pair for the case of two transducer and two pairs in the case of four transducers). We utilized the transducer characteristics of the ATL L7-4 transducer (128 elements). In all simulations, a homogenous medium was assumed. The normalized acoustic pressure profiles of these transducer arrangements were compared in FIG. 3.c. The focal size, characterized by full-width half-maximum (FWHM) of pressure profile generated by a full-ring array indicates a significant narrowing and symmetry compared to different arrangements of linear array transducers. These FWHM values in horizontal (x-direction) are 10.2, 1.8, 1.5, and 1.2 mm; and are measured as 1.8, 2.1, 1.5, and 1.2 mm in vertical (y-direction) for scenarios listed as i-iv.

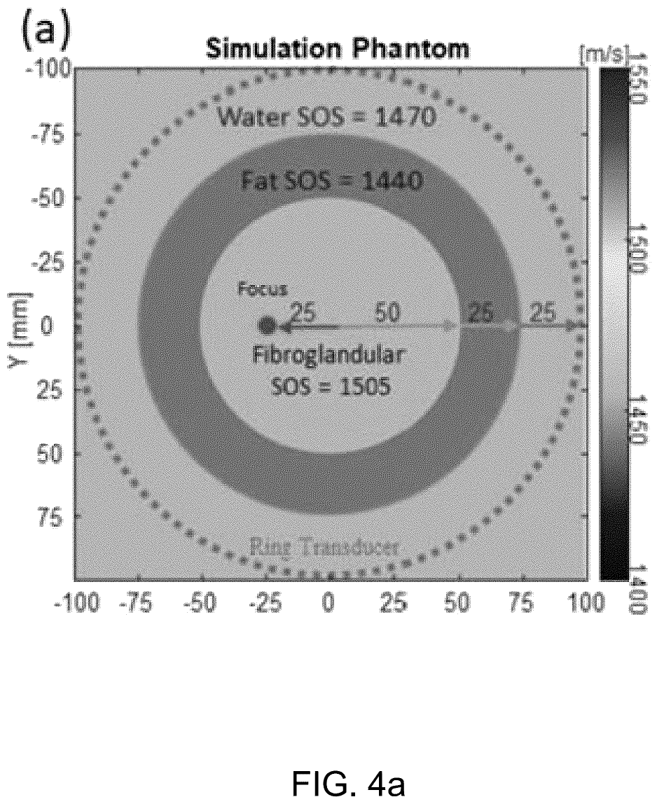

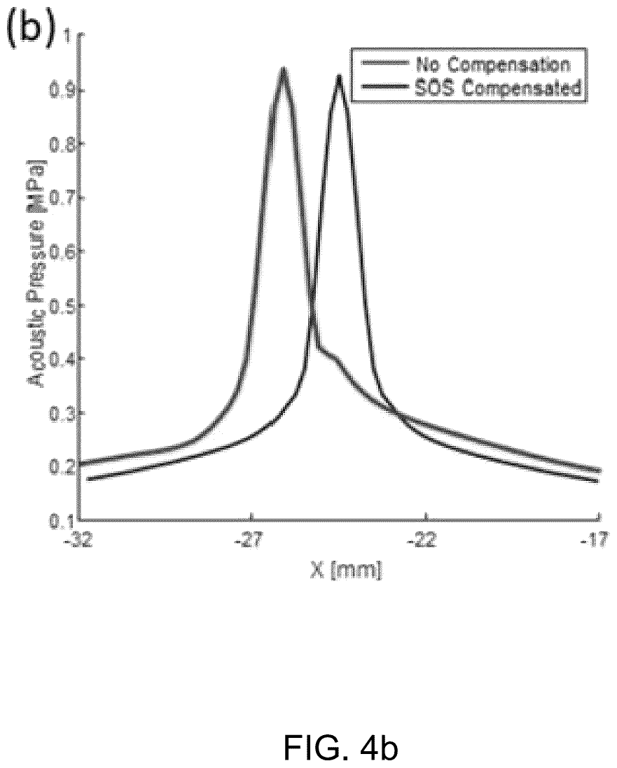

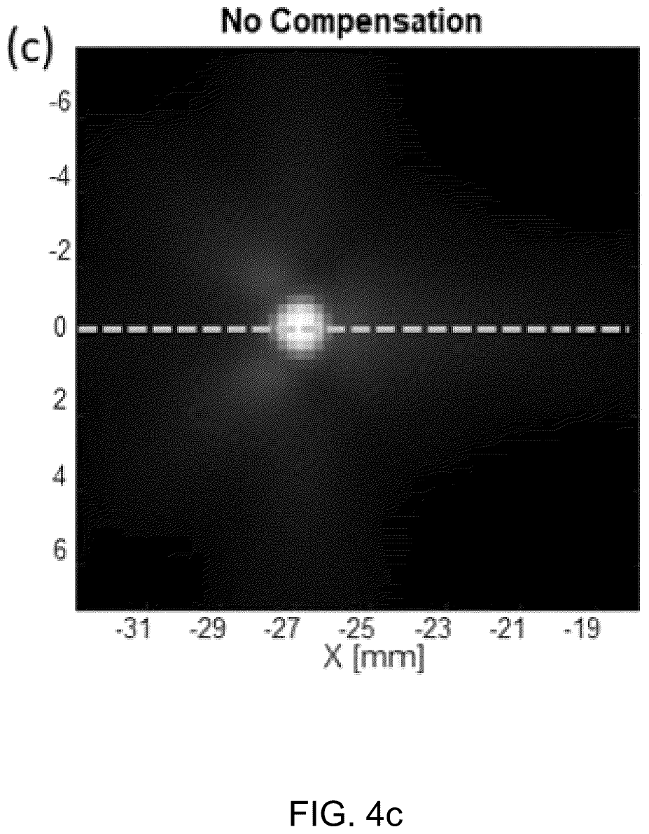

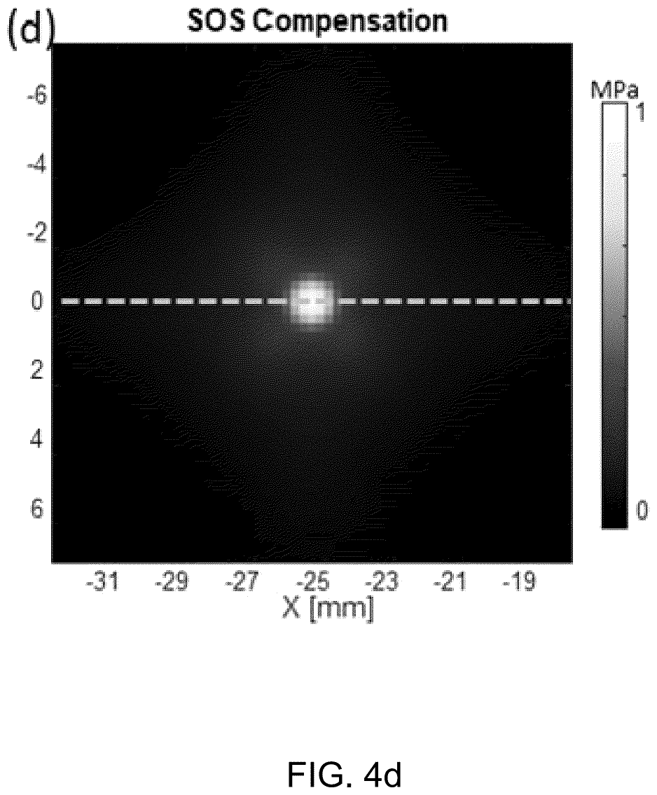

FIG. 4 depicts UST-assisted TR focusing through aberration correction. (a) TR focusing without SS correction; (b) TR focusing with SS correction, showing an improvement in localization and the focal size.

A major limiting factor in TR acoustic focusing is the medium heterogeneity that causes phase and amplitude aberrations and focusing imperfections. UST-guided MHTh system offers a solution for aberration correction and accurate TR focusing through having SS and AA tissue maps. FIG. 4 demonstrates the effect of SS compensation in TR focusing. An identical ring-array transducer was used to induce the heat in a 10 cm diameter numerical breast mimicking phantom, which for simplicity comprises two 5 cm thick layers of fat and fibroglandular. The focus was placed off-center and at a depth of 5 mm (interface of fat and fibroglandular tissue). The focal spot and profile is analyzed when a uniform sound speed was used for TR focusing (FIG. 4a), and when priori knowledge of SS (through UST) was used to provide aberration-free TR focusing (FIG. 4b). Results demonstrated a significant distortion removal on the focal point, when SS correction was done.

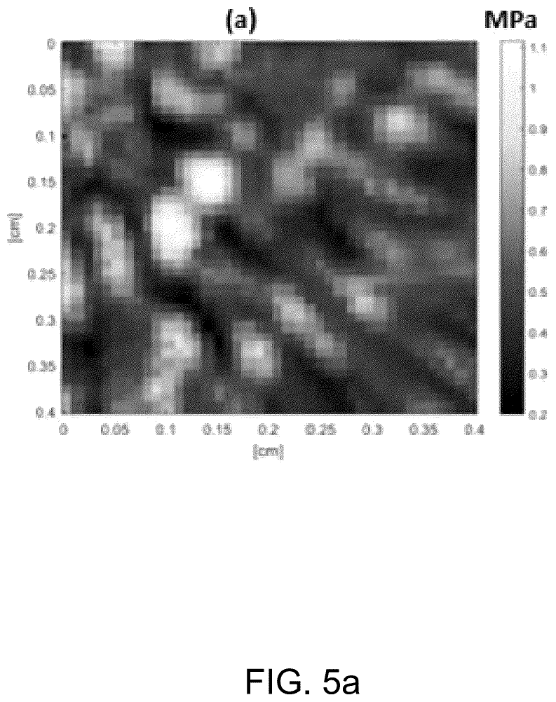

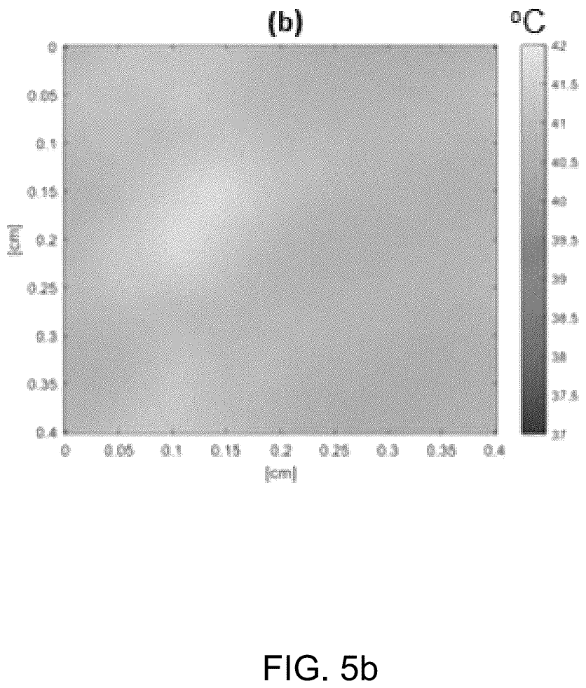

FIG. 5 depicts (a) Focal acoustic pressure measured in a 4×4 mm near the focal point, 2 cm off center of the ring transducer. (b) conversion of the acoustic pressure to heat using a 600/100 on/off cycles for 30.1 seconds, reaching the maximum temperature of 42 °C.

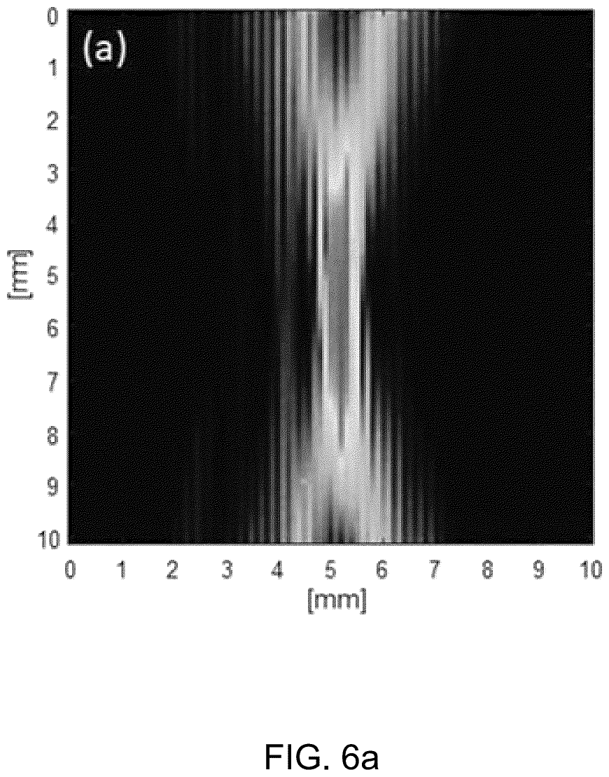

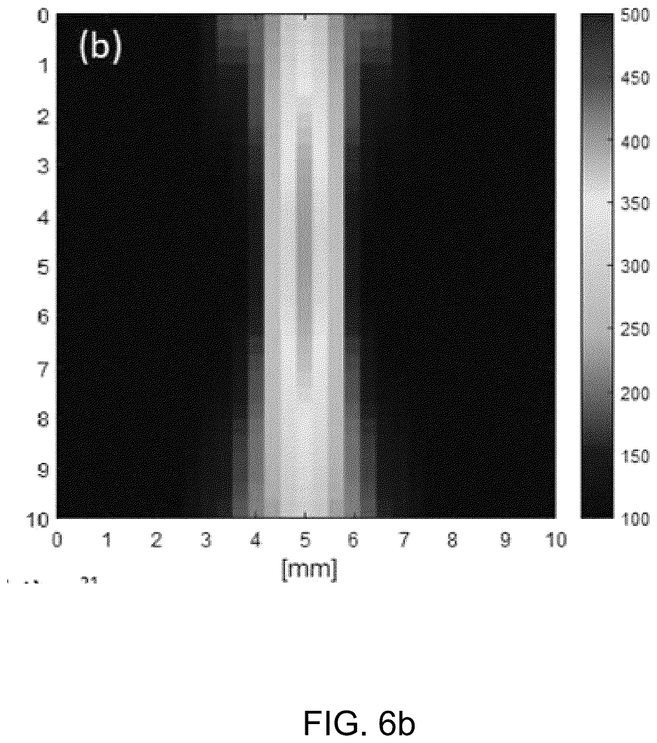

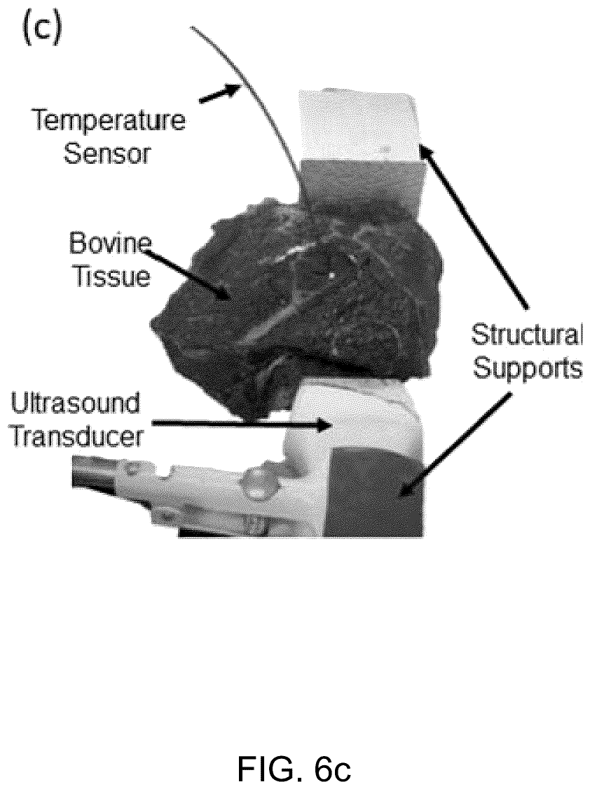

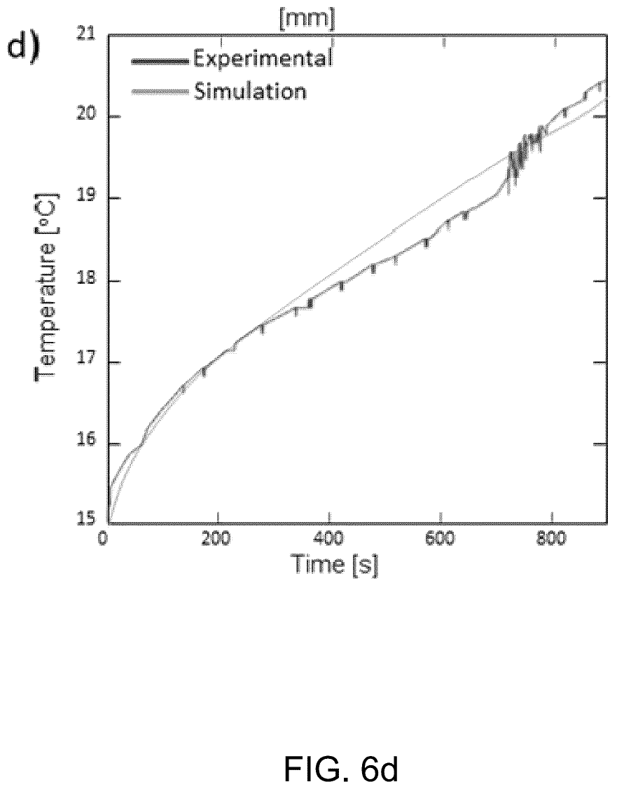

FIG. 6 depicts (a.,b) hydrophone measured, and simulated pressure field generated by phased array P4-1 transducer. (c) experimental setup to measure temperature rise in bovine muscle. (d) Experimentally measured temperature rise. (e) acoustic pressure simulated in bovine muscle (assuming homogenous medium). (f) in-silico temperature rise in bovine muscle based on acoustic pressure in panel e, and after 15 minutes of focused acoustic exposure.

Ring-array to generate MHTh-Preliminary data: Our existing UST system is mostly designed for UST imaging, where one or a very few elements transmit and the received signal on all ring-array elements is used to reconstruct UST SS, AA, and reflection maps. A feasibility test was conducted where a TR focusing was applied on 11 (out of 256) equally distanced transmit elements operating at 1.5 MHz. A needle hydrophone was used as both a focusing target and an acoustic sensor to directly measure the intensity of the field at the focus, which was set to be at a 2 cm off-center. A map of the maximum absolute pressure (|p(r,t)|) over a range of positions r was measured (FIG. 5a), which shows two peaks of approximately the same magnitude but separated by 0.64 mm (~ ½ wavelength at 1.5 MHz). The separation between the peaks is on the order of the diameter of the needle hydrophone and could be due to different sectors of the transducer focusing on different parts of the hydrophone. However, the field focusing in a relatively small region is detected through hydrophone measurements. Using the measured |p(r,t)| and with tuning duty cycle of 600/100 msec ON/OFF and running a bioheat simulation for 600 seconds, leads to the temperature map shown in FIG. 5.b. While the temperature within the target reached 42 degrees, due to the small number of transmit elements and imperfection of acoustic pressure focusing, some temperature rise outside of the target was observed. However, it is anticipated that by using a larger number of elements (as shown in FIG. 3b), a better focusing and thus targeted heating is easily achievable. In order to test and validate the bioheat simulation results with experimental results, we conducted an experiment in which a low frequency phased US transducer (P4-1, Philips-Center frequency of 2.5 MHz) was used to generate heat within a focal location in a bovine tissue. The focal point was set at 3 cm away from the transducer. A filed scan was performed using a small footprint needle hydrophone (HNA-0400, ONDA) to measure the acoustic field intensity for in silico studies (FIG. 6a). Given the difficulty of scanning the field in tissue, it was done in a water bath. To avoid the direct deposition of heat on the temperature measurement device, we utilized an ultra-thin fiber optic temperature sensor (FIG. 6c) with a fiber diameter of 200 μm (TS2, Micronor Sensors), which is much smaller than the acoustic wavelength).

FIG. 6 depicts (a, b) hydrophone measured, and simulated pressure field generated by phased array P4-1 transducer, (c) experimental setup to measure temperature rise in bovine muscle, (d) Experimentally measured temperature rise, (e) acoustic pressure simulated in bovine muscle (assuming homogenous medium), (f) in-silico temperature rise in bovine muscle based on acoustic pressure in panel e, and after 15 minutes of focused acoustic exposure.

FIGS. 6a and 6b compare the simulation and measured acoustic pressure field scans which demonstrates the proper focusing of the phased array transducer and a high correlation between the expected field pattern in simulation (FIG. 6b) and experiments (FIG. 6a). The heating experiment in bovine muscle was done when the US transmit waveform was set to the duty cycle of 1.5% (3 msec on, 197 msec off) given the limitations of existing US system. This will be further modified using a high power HIFU equipped module from Verasonics® which is enabled to provide longer tonebursts. Experimental results measured a temperature rise of 4.6 degrees Celsius from the room temperature baseline (14 °C). The simulation results showed a 5.4 °C (FIG. 6d) temperature rise. The higher temperature rise in this feasibility study is due to a not perfect TR focusing of the phased array transducer in heterogenous bovine muscle (aberrations) and attenuation of the field, which led to a lower pressure field in the focal spot compared to the values used for simulations. It is worth mentioning that the ring-array heating will not face these severe issues due to phase aberrations, as discussed earlier. In summary, a good agreement between experimental and in silico studies to predict the tissue heating is observed (FIG. 6d).

Disclosed is an all acoustic theranostic system for enhancing breast cancer treatment. A relatively simple and compact system will provide accurate tumor heating and improve outcomes of cancer treatment in a mouse model. This is supported by our promising preliminary studies and the vast body of literature that has shown the effectiveness of MHTh in improving outcomes. A UST-guided MHTh system is augmented by treatment planning software, testing and characterizing the developed system on phantoms (where heating can be directly measured) and by comparing treatment outcomes in mouse models relative to unheated controls. It is expected that our system provides sufficient (~42 °C) uniform, and strongly localized heating (margin errors +/−0.5 cm), (ii) UST SS measurements are accurate to monitor the process of tissue heating and assure remaining within the MHTh acceptable range; and (iii) that it improves treatment outcomes in mouse models relative to controls (no MHTh).

Continued work is carried out via the following aims: in Aim 1, the optimum hardware parameters of the all-acoustic UST-guided MHTh system are determined using a set of in-silico studies. These hardware parameters are used to manufacture a suitable ring-array transducer and the UST acquisition and MHTh waveform generators electronics. In addition “treatment planning software” is included in the present invention, in which the sequential procedure of imaging/heating/monitoring is implemented. Aim 2 experimentally validates and characterizes the performance of the developed UST-guided MHTh and the MHTh planning software in a set of anthropomorphic breast phantoms and phantoms made out of excised tissue/tumor and synthetic breast mimicking material. Key performance parameters including time-to-heat, MHTh stability, MHTh uniformity, and accuracy of UST thermometry are characterized. Finally, in Aim 3, the performance of the system is studied in a triple-negative BrCa (TNBC) model by: (a) studying the effects of MHTh on key biological/physiological parameters that are known to dictate the efficacy of ChT, RT, and ImT therapies (blood flow, vascular dilation, and tumor oxygenation). Then we further characterize the performance of the developed system on treatment outcome of a set of therapeutic combinations that are designed based on standard-of-care (SOC) treatment of TNBC.

Aim 1: Design, optimize, and implement a clinically translatable all-acoustic temperature controlled MHTh system. Objectives: (a) To study the effect of ring-array physical parameters on MHTh generation and determine an optimum setting of the system; (b) to develop a MHTh sequential planning software for a temperature-controlled automatic acoustic parameter configuration based on UST-based lesion detection and targeting.

Aim 1.1: In silico study of ring-array UST system parameters for optimum temperature controlled MHTh. Through a set of simulations, we will study the effect of physical characteristics of the ring-array transducer on MHTh and thermometry using quantitative evaluation metrices as follows. Table 2 summarizes the key hardware parameters (both ring-array transducer and backend electronics hardware) to be studied for finding an optimum set of parameters to manufacture UST-guided MHTh hardware.

TABLE 2
In-silico optimization study parameters to determine the practical
and optimized MHTh/UST theranostic hardware (ring array transducer,
waveform generation and acquisition parameters)

| Parameter | Rationale | Values |
|---|---|---|
| Center frequency (f₀) | MHTh: Center frequency will have impact | 0.5, 1, |
| | on penetration depth, focal spot size, | 2, 3 MHz |
| | heating time, and heating pattern 1, 1.5, 2, 2.5 MHz | |
| | UST: f₀ affects spatial resolution of SS | |
| | (temperature), AA, and reflection mode images, | |
| | penetration depth | |
| # of elements in | MHTh: N will affect the focal pattern and focal | 64, 128, |
| the ring-array (N) | intensity, thus temporal characteristics of heating | 256 |
| | UST: Spatial resolution and accuracy of | |
| | SS measurement is affected by N. | |
| MHTh TX waveform | MHTh: T contributes to the speed of heating | 100, 500, |
| duration (T) | UST: Duty cycle in combination with T will | 1000, 2000 |
| | determine the time for interleaved SS imaging | msec |
| | (thermometry) | |
| MHTh TX waveform | MHTh: Duty cycle will determine the speed of heating | %29, %40, |
| duty cycle | UST: Duty cycle in combination with T will determine | %60, %80 |
| | the time for interleaved SS imaging | |
| | (thermometry) | |
| MHTh TX peak acoustic | MHTh: Duty cycle will determine the speed of heating | 50, 100, |
| power (P₀) | (as well as safety characteristics of the | 150 kPa |
| | system) | |
| | UST: NA | |

Parameters listed in Table 1, lead to a total of 576 different combinations. Given that we also want to include four different breast densities (according to the BI-RADS reporting system: level 1 (fatty), level 2 (scattered areas of fibroglandular tissue), level 3 (heterogeneously dense), and level 4 (extremely dense), and two different sizes (10 and 16 cm diameter, representing small and large breast), the number of scenarios must be limited. Therefore, we will first do a study on f₀ and N (12 scenarios) for 8 combinations of breast density and size to find an optimum ring-array transducer parameters that leads to the total number of 96 scenarios. In each simulation, two focal spots are selected: (a) superficial: 10 mm deep from the skin; (b) deep: 50 mm from the skin. Given that studies shown that the majority of breast tumors grow at an interface between fat and fibroglandular, we believe that the range 10 to 50 mm covers a large percentage of BrCa and is suitable to study both deep tissue heating (efficacy) and superficial lesions (risk of skin damage). Optimum ring-array transducer parameters will then be used for studying T, duty cycle, and Po, to further optimize the acoustic parameters related to the backend circuitry. The quantitative parameters to optimize the ring-array and hardware include: MHTh heating 3D profile (point-spread-function), heating time for a typical 20 mm in diameter spherical tumor, and the resolution an accuracy of UST thermometry.

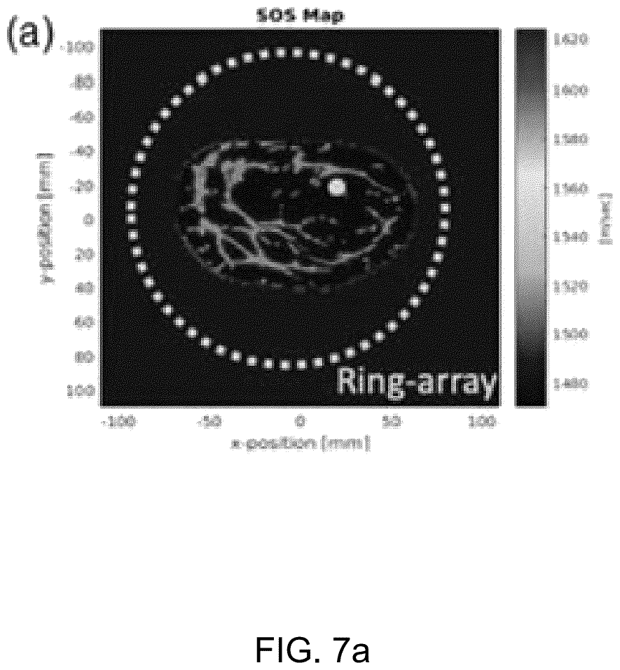

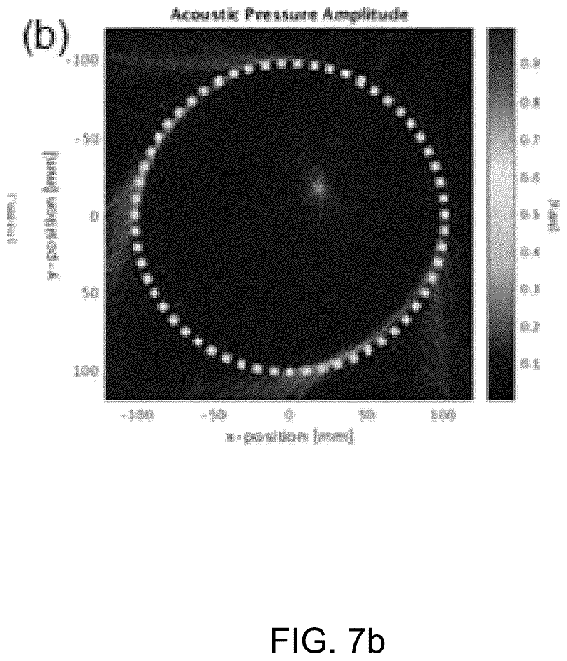

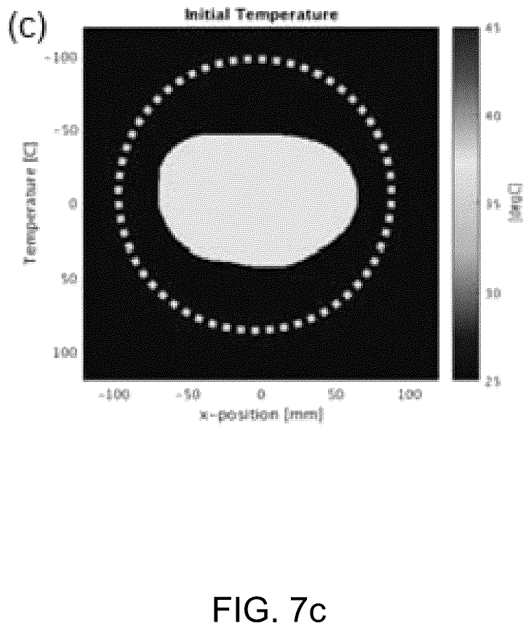

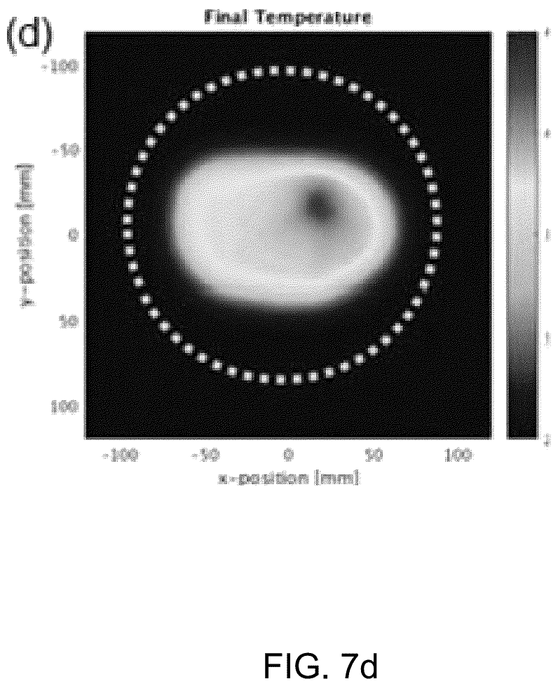

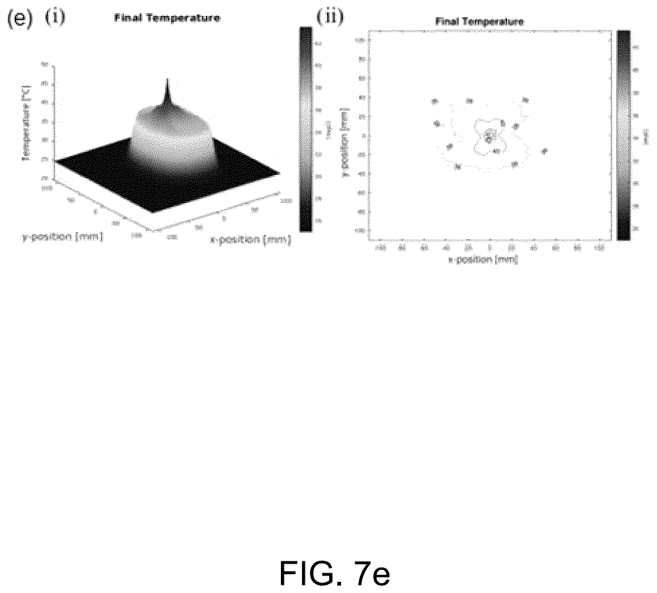

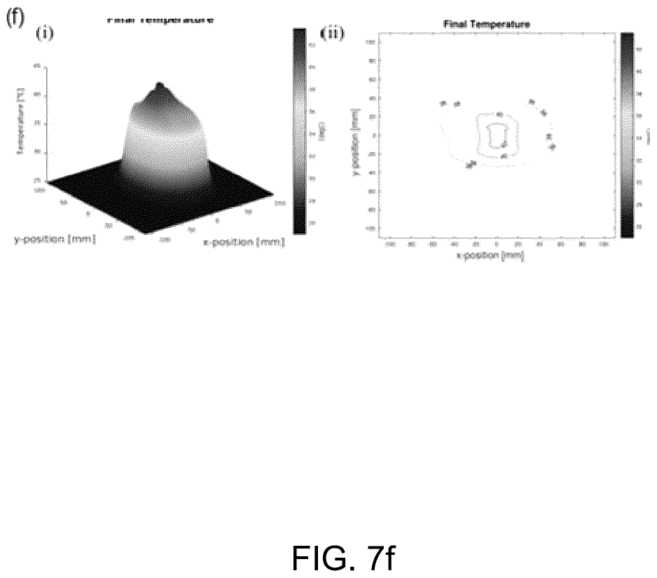

FIG. 7 discloses 2D in-silico MHTh generation results. (a) SS map of a numerical breast phantom including skin, fat, fibroglandular, and tumor. (b) acoustic pressure map after TR focusing. (c,d) pre- and post MHTh temperature maps. (c) Final temperature map for single focus (i) surface plot and (ii) contour plot where regions with temperature rise to 36, 38, 40, 42, and 44 °C are identified. (d) same measurements as in (c), for 8 foci sequential heating. The results indicate more unform and larger tumor volume heating using multi-foci heating strategy.

FIG. 7 depicts 2D in-silico MHTh generation results. (a) SS map of a numerical breast phantom including skin, fat, fibroglandular, and tumor. (b) acoustic pressure map after TR focusing. (c,d) pre- and post MHTh temperature maps. (c) Final temperature map for single focus (i) surface plot and (ii) contour plot where regions with temperature rise to 36, 38, 40, 42, and 44 °C are identified. (d) same measurements as in (c), for 8 foci sequential heating. The results indicate more unform and larger tumor volume heating using multi-foci heating strategy.

An example of an in silico study is shown in FIG. 7. The phantom originated from the Optical and Acoustic Breast Phantom Database (OA-breast), and was composed of skin, fibroglandular, fat, blood vessels and surrounding water components (FIG. 7a). The numerical breast phantom was adapted for our experiment, such that the blood vessels were removed, and a 2 cm diameter spherical tumor was embedded into the center of the breast. Acoustic pressure map after TR focusing at the center of the tumor, and tissue temperature maps before and after applying MHTh for 210 seconds (toneburst of 2000 msec with duty cycle of 90%, element acoustic pressure of 100 kPa) is shown in FIGS. 7b-d.

Since MHTh is often used to assist with treating larger tumors (≥2 cm in diameter), it is important to test the ability of the UST-guided MHTh system in providing heating in larger volumes. This can be done by sequential heating of multiple foci selected within a tumor. FIG. 7f represents the surface plot and temperature contours of produced heat by heating 8 foci points within the tumor to cover a larger volume. In case of the single focus, a peak pressure of ~1.49 MPa was reached at the focus, a peak temperature of 48.85 °C was reached inside the tumor, and 37.1% of the simulated tumor area reached temperatures above 42 °C after 120 s. results indicated that the peak pressure of each of the 8 foci was between ~1.26 MPa and ~1.45 MPa, a peak temperature of 43.82 °C was reached inside the tumor, and 83.5% of the simulated tumor area reached temperatures above 42 °C after 210 seconds.

UST thermometry through SS measurement requires a fast acquisition and fast image reconstruction. While ultrasound waveform tomography is already developed and implemented, we optimize UST using smaller field of view (FOV) to achieve a frame rate of at least 1 UST image per second which will allow for fast enough temperature monitoring during MHTh. We will also study the effect of sparse UST acquisition using a sub-set of ring-array elements and its efficacy on UST spatial resolution and accuracy of measurements.

Aim 1.2: UST-guided MHTh treatment management software. The treatment management software utilizes volumetric multiparametric UST maps (reflection, AA, SS, and stiffness) acquired prior to the MHTh to identify the position and size of the lesion. The software selects the targeted heating volume (automatically based on UST images or manually by the user), the target temperature and the margin for heating (for example, to avoid exceeding in sensitive areas such as closer to a major blood vessel).

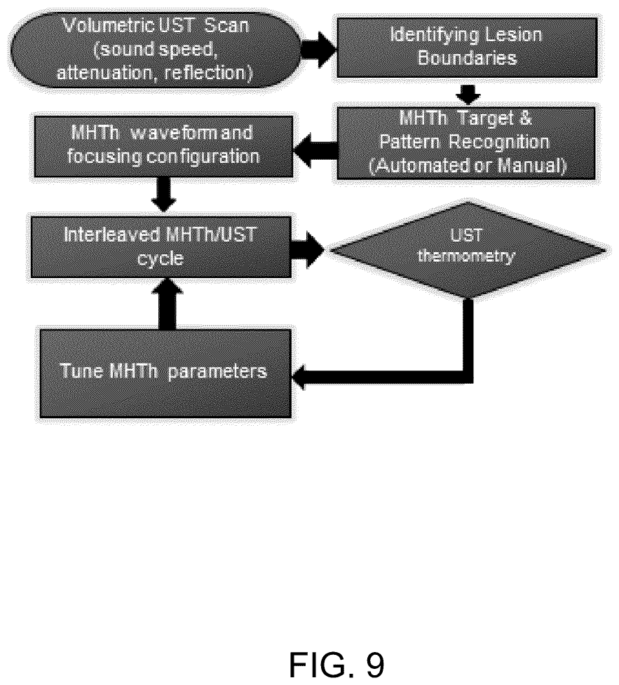

Geometrical characteristics of the lesion (targeted heating volume) will be used to determine the number of foci to achieve uniform heating and to configure aberration corrected TR focusing by utilizing a SS and AA corrected time-reversal forward model from the desired heating pattern, and to deliver uniform heating throughout the target volume, as monitored by UST thermometry. Interleaved UST thermometry during OFF heating cycles will allow for constant monitoring of the temperature. A margin of 0.5 °C will be considered to determine keeping or terminating the MHTh cycle (FIG. 9).

FIG. 9 depicts a schematic of UST-guided MHTh treatment planning algorithm which includes UST lesion identification, MHTh region identification, MHTh aperture and waveform configuration, and UST thermometry feedback.

Uniform heating of a large volume (>4 cm in diameter) tumors may be a challenging problem since it will need a larger number of foci and the sequential focusing will not be suitable to maintain the uniformity of the heating profile. An alternative embodiment (at the cost of need for a larger number of firing element) is to divide the ring-array into multiple sparsed-element ring arrays (for example to 4) and use each sparse-element ring for a foci heating. While smaller number of elements and the sparsity can lead to a sub-optimum focal heating profile, it will increase the uniformity and the heating speed. The uniform heating in a larger volume may not be achievable, especially if the tumor is extended out of the imaging plane. In these cases, the ring-array transducer will need to mechanically scan the tumor volume. Mechanical scan will lead to potential slower temperature increase since the transducer should now generate heat into two physically separate locations in the breast tissue. Upon optimization of the ring-array transducer, the volumetric heating limitations and optimum transducer movement speed to achieve acceptable heating speed will be determined. UST thermometry through SS measurement may require long processing. Fast SS imaging may be employed, and in addition, by limiting the field of view (FOV) for SS imaging within the MHTh target, the SS measurements can be expedited and fit within the allocated time for interleaved MHTh/UST sequence.

Aim 2: Experimentally validate and characterize the performance of the developed system in clinically relevant breast mimicking models.

The objective of this aim is to experimentally validate and characterize the performance of the developed UST-guided MHTh hardware/software in a set of anthropomorphic breast phantoms and phantoms made out of excised tissue/tumor and synthetic breast mimicking material. Key performance parameters including time-to-heat, MHTh stability, MHTh uniformity, and accuracy of UST thermometry will be characterized.

Breast-mimicking phantoms for further validation studies will be developed in house and based on our team's previous experience with mimicking acoustic properties of breast tissue. The phantoms simulate the fat inclusions embedded in fibroglandular tissue. Eight sets of phantoms, simulating different breast density/heterogeneity and sizes will be developed (according to parameters listed in literature). Since it is hard to develop a breast mimicking phantom that fully mimics the scattered dense tissue within the fatty background, we will use averaging and multi-layered phantoms in which we will use an external fat layer, internal fibroglandular mimicking center, and an intermediate layer mimics the mixture of fat and fibroglandular properties with the rations of 20, 40, 60, and 80 percent, representing fatty to dense breast. UST SS, AA, and reflection mode images will be used to characterize the phantoms and assuring the accuracy of the recopies used for making phantoms. For each type of phantom, two breast phantoms with total diameter of 120 and 180 mm representing small/medium, and large breasts will be developed. Each phantom will include 4 cancer mimicking cylindrical lesions, with diameters of 10 mm (small lesions) and 30 mm (large lesion), located at the depths of 20 mm (superficial) and 60 mm (deep) from the surface of the phantom. For simplicity, we assume a single lesion in each MHTh/UST and the lesions of different sizes and depths will be embedded at different vertical positions to avoid interfering with each other's measurements. Parameters representing the performance of MHTh will be characterized for each of the 32 combination of breast density (x4), breast size (x2), tumor size (x2), and tumor depth (x2). These parameters include: (a) Time to reach 42 °C in whole tumor; (b) the uniformity of heating profile in steady-state situation (after the whole tumor reaches 42 degrees); (c) The stability of the MHTh profile characterized by analysis of average, minimum, and maximum temperature over the whole volume of tumor over the treatment time; and (d) the maximum distance outside of the tumor, in which the temperature exceeds 39 degrees (to characterize the localization of heating profile in targeted lesion). All temperature measurements will be performed using ultra-thin fiber optic temperature sensor, similar to a TS2 probe used in preliminary studies. The probes will be embedded into the phantoms during creation of the phantoms and will monitor the temperature at the center of the lesion and at equally-distanced locations of 5 mm apart from each other, from the center of the lesion up to 20 mm outside of the lesion. The temperature measurements will also be compared with UST thermometry to determine the accuracy of UST thermometry during the MHTh procedure. In addition, for each set of experiments, the formation of hot spots on the surface of the phantom will be monitored using UST thermometry and by placement of fiber optic temperature sensors in areas that UST shows elevated temperature. The temperature of the water tank and the phantoms (after enough time to reach equilibrium) at the beginning of each test, will be kept at 37 °C.

To further evaluate the accuracy of UST thermometry, 8 similar breast phantoms will be modified to pass controlled temperature water (ranging from 37 °C to 45 °C degrees, with increments of 0.5 °C) using a piping system through them. Two sets of 5 mm and 20 mm in diameter US transparent tubes will be embedded in phantoms, and at the center of the small and large tumors as in previous studies. UST thermometry measurements will be compared with ground truth (a thermocouple placed in the middle of the tubes). The error difference between UST temperature measurement at each temperature will be calculated.

In addition, safety parameters of the system such as potential heat generation on the skin (or within the tissue) will be carefully monitored.

In addition to testing the performance of the UST-guided MHTh system, we will also examine the hardware stability by monitoring the temperature at the surface of the elements while the system is operating for 60 minutes (expected operation time in pre-clinical and future clinical studies). We will also examine the stability of the ring transducer in terms of acoustic power output prior to conducting each experiment, to avoid any systematic experimental error.

Aim 3: Test and assess the efficacy of MHTh produced by the developed all-acoustic system in a breast-tumor model. TNBC represents ~15-20% of all newly-diagnosed BrCa, and remains the most aggressive subtype with poorest outcome. No molecular targets exist for TNBC, and alternative treatment strategies are urgently needed to improve survival. RT is a SOC therapy in BrCa because RT-induced DNA damage causes direct tumor cell death but also induces immune responses through release of tumor antigens and generation of a favorable inflammatory cytokine milieu from irradiated and dying tumor cells. Consequently, immunotherapy (ImT: checkpoint blockade mAbs) potentiates RT effects by increasing tumor specific T-cell proliferation, improving outcomes over either modality alone. Preclinical studies have shown that combination of RT and anti-PD-1/PD-L1 therapies activate CD8 T cells, reduce inhibitory mechanisms, and induce abscopal effect. While, in human clinical trials (e.g. IMpassion 130 and KEYNOTE-355) overall and progression-free survival in patients with advanced TNBC improved when ChT was combined with blockade of PD-L1 versus ChT alone. PD-L1 blockade in combination with ChT was FDA-approved for patients with early stage and advanced TNBC with high PD-L1 positivity. However, response was only positive for those with high PD-L1 expression (>10) which does not address >50% of the TNBC patient population. Clinical trials using checkpoint inhibitors against PD-1 and PD-L1 as single agents have shown promising results in BrCa; however overall response rates for TNBC of 18.5% (PD-1) and 24% (PD-L1) suggest that additional methods are required to significantly improve tumor control. Our aim is to use MHTh as one such method. By using MHTh to increase vascular flow, our methodology overcomes limitations of systemic delivery for ImT mAbs that directly target RT-induced tumor microenvironment, whilst also enhancing the cytotoxic effects of RT on DNA to increase cell killing. Also, RT contributes to immune system activation via cross priming, via tumor antigen release and activates dendritic cells (DCs). These DCs migrate to the regional lymph nodes and encounter and activate tumor-specific cytotoxic T cells. The hypothesis to be tested in this aim is that MHTh enhances the efficacy of RT in combination with ChT/ImT (blockade of PD-1/PD-L1 signaling).

Animal model and preparation: Subcutaneous, and later orthotopic, 4T1 tumors (and later 4T07 tumors) will be investigated. The 4T1 tumor is a good experimental animal model for human BCa. 4T1 tumor cells grow in the mouse mammary gland and metastatic disease develops spontaneously from the primary implanted tumor. Also, the primary tumor can be surgically removed, so that metastatic disease alone can be studied. 4T1 tumors have been widely used for ImT studies targeting the activation of tumor-specific CD4⁺ and CD8⁺ T lymphocytes. 4T1 cells (ATCC CRL-2539) are grown in vitro in RPMI-1640 supplemented with 10% FBS with 2 mmol/L L-Glutamine and antibiotics, prior to implant in BALB/c mice. We remain cognizant that BALB/c mice, which are required for 4T1 cells, are prone to radiation-induced mammary carcinoma formation and are radiosensitive. Initially, subcutaneous (or intradermal) tumors implanted in the leg will be used to establish the optimal heating parameters; this tumor location provides the most pragmatic and simplest tumor model to test MHTh in combination with RT. Then, later studies will use orthotopic tumors in the mammary fat pad when ImT is added, and metastatic studies are undertaken with RT, MHTh and ImT. Tumors will be established in 6-8 week-old BALB/c female mice by injection of 1×10⁶ cultured tumor cells (in 100 μL Matrigel), using established tumor implantation procedures.

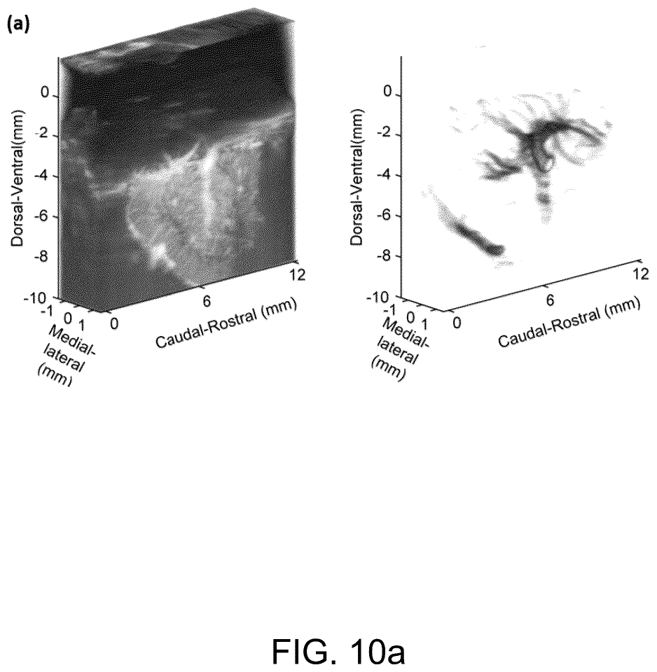

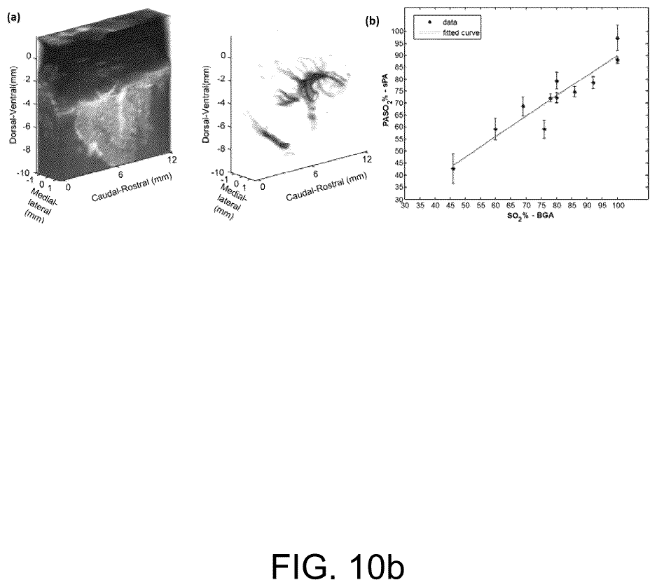

FIG. 10 depicts (a) 3D vasculature map: (left) Power doppler images stacked in the medial-lateral dimension to form a volume in one ferret, viewed from the caudal perspective. (right) displayed the raw blood flow signal intensity. (b) Blood SO₂ measure by sPA vs. gold standard blood gas analysis.

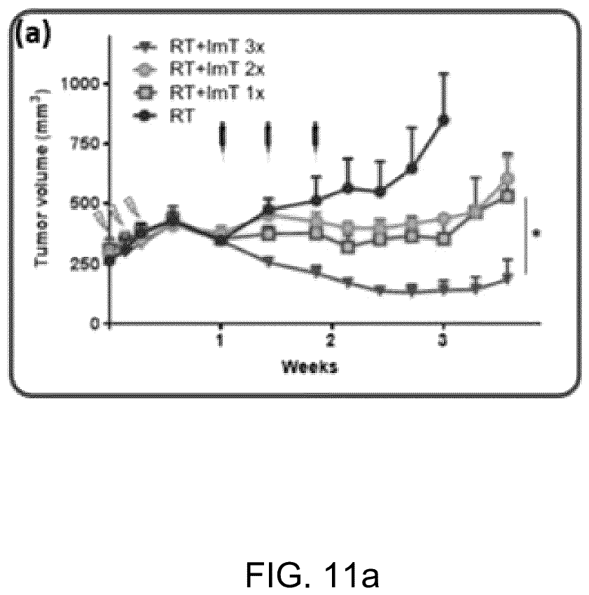

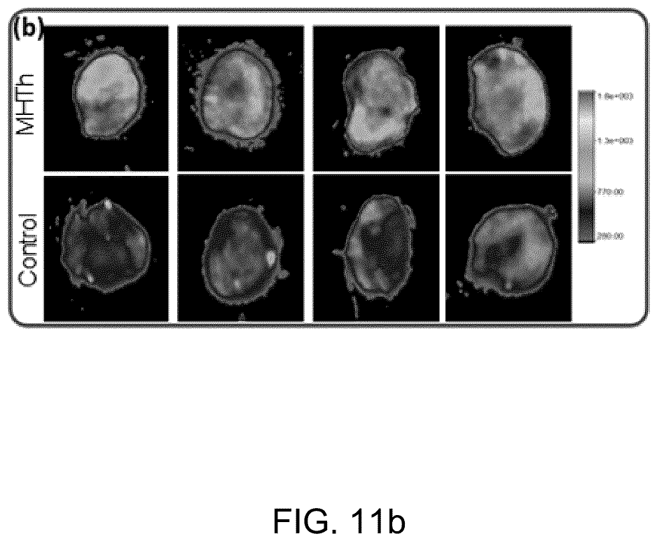

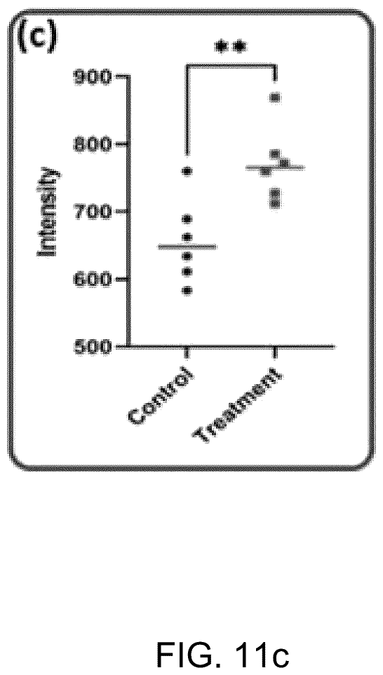

FIG. 11 depicts (a) Representative images showing higher Doxil accumulation in the MHTh-treated (top) versus untreated tumor; (b) Quantification of fluorescence intensity demonstrates statistical difference in uptake between treated and control 4T1 tumors. (c) Tumor volume as a function of time for 4T1 tumors treated with 8Gyx3 and either one (1×), two (2×) or three (3×) doses of immunotherapy. RT+ImT improved response above RT alone.

Aim 3.1, To examine the effect of MHTh on vascular dilation, blood flow (perfusion), and tumor oxygenation. We use n=10 animals for this study and once the tumor size reached 8-10 mm in diameter, we will place the animals in a breast mimicking holder (multi-layer phantom) and apply MHTh/UST sequences. We plan to keep the tumor temperature at 42 degrees for 60 minutes. We will also acquire 3D volumetric (mechanically scanned) Doppler US for measuring total blood flow and also vasculature size using a separate US imaging system, equipped with a high frequency animal studies probe (L22-14v). We will acquire the blood flow information when the tumor is placed inside a 37 °C water tank, and during a 60 minute period heating every 5 minutes. Upon finishing the MHTh procedure, we will continue to monitor vasculature and flow for 4 hours and at time increments of 30 minutes. We will continue the measurements for 72 hours then at increments of 6 hours. At the same time and using the same high frequency US transducer, we will acquire spectroscopic photoacoustic (sPA) volumetric images of the tumor at 2=750 nm, 780 nm, and 800 nm and will use our previously reported spectral unmixing method to provide volumetric tissue SO₂ maps. sPA measurements will be performed at the same time points as volumetric Doppler blood flow and vascular measurements will be performed. Our previous sPA oximetry showed accuracy of revealing blood oxygenation over a wide range of SO₂ between 46 and 100%. Expected outcome: A clear understanding of the changes in important tumor microenvironment parameters affecting ChT, RT, and ImT will be quantitatively measured. This data can be used to further optimize.

Aim 3.2: To examine the effect of MHTh on anti-tumor response in combination with standard of care (SOC) therapies in TNBC model. Animals will be randomized to treatment groups. After the tumor volume has reached 100-150 mm³, tumors will be treated with MHTh (starting with 42.5 °C) immediately before (20-40 mins) or after RT (up to 60 mins) using the developed MHTh/UST device, optimal duration of MHTh and temperature will be defined. During MHTh/UST experiments, tumor bearing animals will be placed within a phantom comprising a two layer fat-fat/dense tissue (50%-50%) and at the depth of 30 to 50 mm, as described in Aim 2, to mimic the deep lying breast tumors. RT will be a sub-curative single dose (SD: 10-20Gy) or more clinically-relevant fractionated RT (F-RT: 8×3Gy; 5×4Gy). RT will be focally delivered to the whole tumor volume under CT-image guidance with the MuriPlan software within XStrahl SARRP X-irradiator (small animal radiation research platform), as per our established SARRP RT procedures. This methodology ensures precise RT tumor targeting, and sub-curative doses are used because the efficacy assay assesses treatment-induced tumor growth delay, while also allowing the evaluation of additional cytotoxic combinational therapies to extend tumor growth delay towards tumor cure. The MHTh/UST prototype will be outfitted with nose cones to deliver isoflurane as an inhalant anesthetic. Small (<100 mm³) and large 4T1 tumor burden (300-500 mm³) will be compared to determine how MHTh can improve response to treatment as a function of tumor size and tumor hypoxia. Mice are positioned so that their hind legs bearing 4T1 tumors are heated with MHTh/UST prototype. Based on published literature, we will begin with 42.5 °C for 30-40 mins during RT and determine the Thermal Gain Factor, and this will also be compared to MHTh before or after RT using the same heating regimen to determine optimal sequencing. Tumors will be given MHTh, IMT/ChT treatment (anti-PD-L1+paclitaxel) with and without MHTh, RT with and without MHTh and multiple treatments together (MHTh+IMT/ChT+RT). Tumor response will be evaluated by assessing tumor volumes every two days using digital calipers, calculating tumor volumes using elliptical volume calculations. The feasibility of this irradiation methodology using the 4T1 tumor challenge model has been demonstrated. On days 3, 6, and 9 after-RT and MHTh, mice will be injected intravenously (i.v.) with 30 μg of PD-1mAb or PD-LImAb alone (n=10 per group). Infiltration of immune effector cells (e.g. CD8 TILs, CD4 T cells, NK cells, dendritic cells and M1 macrophage), immune suppressive cells (e.g. M2 macrophages, Tregs) and upregulation of PD-L1 checkpoint will be determined via immunohistochemistry staining and flow cytometry. Release of effector cytokine molecules in mouse sera and tumors will be investigated and quantified via ELISA assays. For metastatic studies, overall animal survival will be assessed from metastatic disease by evaluating animals for signs of morbidity. To determine the abscopal responses (ARs) induced after RT, MHTh, ImT and combinations, 6-8-week-old mice will be injected subcutaneously on the right and left flanks (bilateral flank tumor) with 2×10⁵ 4T1 tumors cells. After tumor volume has reached 100-150 mm³, one of the two bilateral tumors will be selectively irradiated with single dose (SD: 20Gy) and fractionated RT (F-RT: 8×3Gy; 5×4Gy). The optimal heating parameters as defined in our prior experiments will be used. Animals (n=10 animals per group) will be injected i.v. ImT as described above. 24 hours after the last drug treatment, animals will be anesthetized, and irradiated tumor will be surgically removed and skin sutured. Contralateral non-irradiated tumor growth will be evaluated by assessing tumor volumes every 2-3 days using digital calipers and tumor volume will be calculated. Alternatively, single primary tumor will be implanted after irradiation and MHTh and/or ImT, the development of lung metastasis will be assessed by micro-CT imaging. At the time of necropsy, lung weight and tumor volume will be evaluated and along with adverse toxicities by animal weight. Lungs will be fixed in formalin and embedded followed by staining with hematoxylin and eosin or homogenized to generate a single-cell suspension and CD8⁺ T cells will be stained with CD8a BV786 (eBioscience) antibody for flow cytometry. Three days after the last treatment, animals will be euthanized, blood will be collected for cytokine analysis, and tumors, lungs, liver and spleen will be weighed and processed for toxicity evaluation. In our pre-clinical mouse model, we will study the changes of tumor blood supply and tumor oxygenation in response to MHTh, to provide baseline data for an optimum combination of MHTh with ImT and RT sequence and timings. We will also examine the effects of MHTh on anti-tumor immune response in combination with SOC in TNBC model. Specifically, we will determine if MHTh facilitates improved T cell immune infiltration into the tumor with inhibition of tumor growth. As proof-of-concept, we have investigated the potential of MHTh to increase accumulation of PEGylated liposomal doxorubicin (Doxil™), a known DNA damaging agent, in TNBC 4T1 syngeneic tumors. Doxil was administered intravenously (i.v.) immediately prior to exposure to a prototype MHTh chamber with temperature adjusted to 42 °C. The tumor grown on the hind limb was immersed in the 42 °C water. Because of Doxil's inherent ability to fluoresce, its tumor accumulation was measured using fluorescence intensity. 4T1 tumors treated with MHTh showed increased fluorescence versus Doxil-administered tumors without MHTh (FIG. 12b, c). Overall tumor response will be investigated to determine the effect of MHTh when combined with SOC treatment comprised of (i) ImT and ChT, or (ii) external beam radiotherapy, or (iii) combined ChT/ImT with RT. Further, we will be assessing how MHTh can overcome large tumor burdens that are less responsive to treatment.

Statistical considerations: Each group consists of 5 mice and will be repeated twice for n=10. Linear mixed-effects (LME) models will be fitted to describe changes in the primary outcome: tumor volume over time. Comparison of tumor volume over time by group will be performed by using an adjusted area-under-the-curve (aAUC) method. As an illustration of statistical power, we conducted Monte Carlo simulation via 10,000 repetitions for 5 mice per group using the ratio of aAUCs (aAUC_{RT+ImT}/aAUC_{RT alone} and aAUC_{RT+ImT}/aAUC_{ImT alone}). The set-up for the simulation is based on the mean of relative change of tumor size at the end of the experiment, with a predicted change based on preliminary data of approximately 2 (i.e., 200% increase) for RT only and 1.25 for RT+MHTh, with 0.25 standard deviation for each group. Statistical power to test whether the ratio of aAUCs is greater than 1 are 94.1% based on 95% one-sided confidence interval of the ratio of aAUCs between RT alone and RT+MHTh. Boxplots and parallel coordinate plots will also be used to describe the above tumor growth over time by group. Histological biomarkers will be calculated and compared across groups using Kruskal-Wallis tests. Post-hoc pairwise comparisons will be performed by Student t-test or Mann-Whitney U test with Bonferroni multiple test adjustment. Body weights will be measured once a week and we will characterize changes of body weight using polynomial regression. For the metastasis studies, similar statistical analysis will be used to compare the treatment groups. Additionally, organ weight, tumor volume, and cytokines at sacrifice will be compared across groups using Kruskal-Wallis tests. Tests will be two-sided and findings will be considered statistically significant if p-value <5%. Analysis will be performed using R by the project statistician.

Irradiations: Dosimetric calibration of x-ray irradiators is accomplished using AAPM TG 61 Task Report on 40-300 kV X-ray Dosimetry in Radiotherapy and Radiobiology by Dept. Medical Physicist. Ionization chambers are used for irradiator output calibration and HVL measurement and calibrated yearly by the AAPM Accredited Dosimetry Calibration Laboratory. XV and GaF Chromic EBT2 films are used to measure the field size, the relative output of the small RT field/cones, the depth dose and the dose profile.

Potential problems & alternative strategies: (a) We have disclosed preclinical experiments to evaluate the heating potential of the MHTh/UST device, and this will be achieved by comparing the efficacy of established cytotoxic therapies (RT, ImT, ChT, combinations) used in the treatment of BrCa in the absence and presence of heat, we anticipate heating will improve efficacy. However, we will compare the efficacy of each therapy alone, then in combinational regimens, as we remain cognizant that multiple therapies can be toxic unless controlled for and can be problematic; the inclusion of localized heat and localized RT should minimize off-target toxicities. We have disclosed experiments with 4T1 tumors as an ideal model for TNBC, but will confirm the efficacy of optimal treatment parameters in tumor BrCa models (4T07) and rapidly-growing Lewis Lung Cancer in C57BL/6, to provide a different mouse strain also. These experiments ensure our observations with 4T1 have wider applicability to other tumor models. (b) Breathing motion of the animal can cause the tumor (targeted region for MHTh) to move in and out of treatment zone and thus reduce the MHTh efficiency. While the location of the tumor made it less affected by breathing motion, we consider using a viscous gel to put around the tumor to better restrain the animal and reduce the motion.

The present invention may also be used for thermal excitation of temperature-sensitive liposomes filled with chemotherapeutic agents (e.g. doxorubicin, alvespimycin). Utilizing these thermosensitive carriers, has been shown to produce a 3 to 25 times increase in doxorubicin/alvespimycin concentration at the tumor site. Such thermosensitive chemotherapeutic agents such as heat activated liposomes (i.e. ThermoDox) have shown tremendous potential to enhance the chemotherapy procedures.

Other uses of the present invention, include, but are not limited to, breast cancer treatment; other types of cancer such as, but not limited to, liver, prostate, thyroid, and even brain (using a low frequency UST system) may also benefit from the system and methods of the present invention.

It is, therefore, apparent that there has been provided, in accordance with the various objects of the present invention, a system and method for ultrasound tomography guided localized mild hyperthermia using a common closed geometry transducer.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, claims and drawings appended herein.

What is claimed is:

1. A system for ultrasound-guided localized mild hyperthermia comprising a closed geometry transducer, an imaging modality, a thermometry modality, and a therapeutic modality; wherein the imaging modality, the thermometry modality and the therapeutic modality use the closed geometry transducer in common.

2. The system for ultrasound-guided localized mild hyperthermia of claim 1, wherein the closed geometry transducer is a ring array transducer.

3. The system for ultrasound-guided localized mild hyperthermia of claim 2, wherein the ring array transducer comprises 64 elements.

4. The system for ultrasound-guided localized mild hyperthermia of claim 2, wherein the ring array transducer comprises 128 elements.

5. The system for ultrasound-guided localized mild hyperthermia of claim 2, wherein the ring array transducer comprises 256 elements.

6. The system for ultrasound-guided localized mild hyperthermia of claim 2, wherein the ring array transducer comprises multiple sparsed element ring arrays.

7. The system for ultrasound-guided localized mild hyperthermia of claim 1, wherein the therapeutic modality comprises the application of therapeutic acoustic signals to target tissue of a patient wherein the therapeutic acoustic signals are configured to cause heating of a specified portion of the target tissue of the patient.

8. The system for ultrasound-guided localized mild hyperthermia of claim 7, wherein the application of therapeutic acoustic signals is controlled by a microprocessor.

9. The system for ultrasound-guided localized mild hyperthermia of claim 7, wherein the therapeutic acoustic signals are applied using time reversal (TR) focusing.

10. The system for ultrasound-guided localized mild hyperthermia of claim 9, wherein the time reversal (TR) focusing comprises aberration correction.

11. The system for ultrasound-guided localized mild hyperthermia of claim 1, wherein the imaging modality comprises ultrasound tomography (UST).

12. The system for ultrasound-guided localized mild hyperthermia of claim 1, wherein the thermometry modality comprises ultrasound tomography (UST).

13. The system for ultrasound-guided localized mild hyperthermia of claim 1, wherein the thermometry modality further comprises tomographic temperature mapping.

14. The system for ultrasound-guided localized mild hyperthermia of claim 7, wherein the application of therapeutic acoustic signals to target tissue of a patient is controlled by the thermometry modality and the imaging modality.

15. The system for ultrasound-guided localized mild hyperthermia of claim 14, wherein the control is with a microprocessor.

16. The system for ultrasound-guided localized mild hyperthermia of claim 1, further comprising ultrasound tomography guided mild hyperthermia treatment management software.

17. The system for ultrasound-guided localized mild hyperthermia of claim 16, wherein the ultrasound tomography guided mild hyperthermia treatment management software comprises ultrasound tomography lesion identification, mild hyperthermia region identification, mild hyperthermia aperture and waveform configuration, and ultrasound tomography thermometry feedback.

18. The system for ultrasound-guided localized mild hyperthermia of claim 1, further comprising a thermosensitive chemotherapeutic agent that is acted on by the therapeutic modality.

19. A system for ultrasound-guided localized mild hyperthermia comprising a closed geometry transducer, an imaging component, a thermometry component, and a therapeutic component;
wherein the imaging component, the thermometry component and the therapeutic component use the closed geometry transducer in common.

20. The system for ultrasound-guided localized mild hyperthermia of claim 19, wherein the closed geometry transducer is a ring array transducer.
